(19) United States
(12) Patent Application Publication
(10) Pub. No.: US 2007/0055132 A1
(43) Pub. Date: Mar. 8, 2007

(54) Catheter device

(75) Inventors: Estelle Camus, Erlangen (DE); Thomas Redel, Poxdorf (DE)

Correspondence Address: SIEMENS CORPORATION;INTELLECTUAL PROPERTY DEPARTMENT, 170 WOOD AVENUE SOUTH, ISELIN, NJ, 08830, US

(73) Assignee: Siemens Aktiengesellschaft

(21) Appl. No.: 11/516,142

(22) Filed: Sep. 6, 2006

(30) Foreign Application Priority Data
Sep. 6, 2005 (DE) 10 2005 042 338.8

(51) Int. Cl. A61B 5/05; A61B 6/00

(52) U.S. Cl. 600/407; 600/476

(57) ABSTRACT

Catheter device with an imaging device, in particular for optical coherence tomography, for insertion into a bifurcation region of a vessel of the human or animal body, comprising a main catheter with a balloon arranged thereupon and inflatable via a supply line, for the purpose of occluding the vessel proximal to the bifurcation, with a total of two further occlusion balloons for positioning in one of the two vascular branches in each case distal to the bifurcation being arranged on the main catheter and/or at least one auxiliary catheter, at a distance from the first balloon, such that they can be inflated for the distal occlusion of the respective vascular branch.

### CROSS REFERENCE TO RELATED APPLICATIONS

This application claims priority of German application No. 10 2005 042 338.8 filed Sep. 06, 2005, which is incorporated by reference herein in its entirety.

### FIELD OF THE INVENTION

The invention relates to a catheter device with an imaging device, in particular for optical coherence tomography.

### BACKGROUND OF THE INVENTION

It is possible to obtain high-quality cross-sectional images of vessels, such as coronary vessels for example, with the aid of optical coherence tomography. The use of optical coherence tomography has however hitherto essentially been limited to vascular segments which do not have branchings. The reason for this is that with bifurcations, the blood reflux from the two vascular segments after the branching means the imaging itself would be significantly adversely affected when rinsing with a rinsing agent. This particularly applies to larger vessels. At present there is no possibility whatsoever of extending the uses of optical coherence tomography to the carotid artery or the cerebral vessels for instance, as no catheter system has hitherto existed with which it would be possible to produce the required special conditions for imaging in these areas by means of optical coherence tomography. Occlusion times of up to 10 minutes would be implemented on the carotid artery for instance.

If optical coherence tomography is to be used on bifurcations, the occlusion must be carried out proximal to the branching when known catheter devices are used. A significant quantity of rinsing agent, such as for instance saline solution or CO₂, must be used to display the two vascular branches, in order thus to prevent a reflux of blood from said two vascular branches. Such a massive introduction of rinsing agent puts a significant strain on the tissue supplied by the vessel, particularly in the case of branches of large vessels such as the carotid artery. Furthermore, the occlusion of the blood inflow allows the pressure in the vessel to be reduced from arterial values of approximately 100 mm HG to venous values of 30 mm Hg for instance, so that the anatomy of the vessels representing elastic tubes changes and thus the obtained image is only meaningful to a limited degree.

Pathological changes to bifurcations nevertheless represent an important area for the imaging, but stenoses of this type nevertheless make up approximately 15% of the treated lesions in cardiology for instance. Such stenoses are treated by means of interventions in that by considering the individual vascular geometry, the position and type of stenosis and the vascular pathology, stents, so-called drug-eluting stents for instance, are introduced into the vascular region. This process allows several stents to be used, in which one is introduced into the main branch and one into a branching for instance. Critical spots hereby result at the points at which the introduced stents abut each other, whereupon different techniques such as T-stenting, kissing stents etc. are used.

Furthermore, aneurisms are of importance in the region of bifurcations, with which aneurisms an exact knowledge of the pathology is likewise desirable in order to select the suitable therapy. Here imaging modalities are similarly required, as with the treatment and control of stenoses, said imaging modalities enabling the most artifact-free representation of the therapeutic means possible as well as of the pathological factors.

The use of double balloon catheters is certainly already known for imaging within the scope of optical coherence tomography, with which double balloon catheters, on the one hand, the main branch at a bifurcation, and on the other hand one of the vascular branches after bifurcation can be blocked, in order to reduce the use of rinsing agent. Nevertheless, there still exists the problem of the retrograde blood flow from the vessel which is not blocked, which clearly restricts the possibilities of high-quality imaging.

### SUMMARY OF THE INVENTION

The object underlying the invention is thus to specify a catheter device with an imaging device, in particular for optical coherence tomography, which is designed for insertion into a bifurcation region of a vessel of the human or animal body and is improved in respect of the known catheter devices.

A catheter device as described previously is provided to achieve this object, said catheter device comprising a main catheter with a balloon arranged thereupon which can be inflated via a supply line in order to occlude the vessel proximal to the bifurcation, with a total of two further occlusion balloons being arranged on the main catheter and/or on at least one auxiliary catheter, at a distance from the first balloon, for positioning in each one of the two vascular branches distal to the bifurcation such that they can be inflated for the distal occlusion of the respective vascular branch.

In accordance with the invention, a total of three balloons is thus used, one of which one blocks the main branch of the bifurcation proximal to the branching, whereas the others, which are either arranged on the main catheter and on an auxiliary catheter or both respectively on an auxiliary catheter, block both vascular branches after the branching, in other words distal to the bifurcation.

The return flow of blood from the main branch is hereby blocked on the one hand, and on the other hand the retrograde reflux from the two vascular branches after bifurcation is likewise prevented. The use of a rinsing agent putting a strain on the patient and/or the tissue can hereby be reduced to a minimum.

The further occlusion balloons can be arranged in each instance on an auxiliary catheter, which is designed to be guided in a lumen of the main catheter, if necessary in a common lumen with the other auxiliary catheter. In this case, the two auxiliary catheters are advantageously guided in one or two lumina of the main catheter until just before the bifurcation, in order then to be introduced into the two branches. If the corresponding lumen of the main catheter is large enough, both auxiliary catheters can be guided in a common lumen. The use of two auxiliary catheters is then provided for instance if the main branch of the bifurcation is significantly stronger than the two branches considered individually, if for instance an approximately equal distribution of the blood flow to the two bifurcation branches is provided. In this case, two significantly smaller auxiliary catheters compared with the main catheter can be inserted. Furthermore, the use of two auxiliary catheters is preferable if a difficult vascular geometry is present, in which both vascular branches fork off at a considerable angle as an extension of the main branch for instance.

Alternatively, the first of the further occlusion balloons can be arranged on the main catheter and the second occlusion balloon on an auxiliary catheter, which is designed to be guided in a lumen of the main catheter. Such a design lends itself for instance to the situation in that the main branch is continued in one of the two bifurcation branches with an approximately unchanged size and without any significant change in direction. In this case, an auxiliary catheter with an occlusion balloon for occluding the smaller vascular branch can be guided in a lumen of the main catheter and introduced into the smaller vascular branch, starting from the main catheter. To this end, it is inserted into the vascular branch through an opening in the main catheter, at an angle deviating therefrom.

Finally, both further occlusion balloons can also be arranged on bifurcation branches of the main catheter, for which purpose a precise adjustment of the one main catheter to the individually present vascular geometry is nevertheless required. In this case, the two bifurcation arms of the main catheter would be combined accordingly in order to introduce the catheter into the vascular region for imaging, and bound together for instance and separated spatially from one another afterwards, by undoing a connection for instance, in order to be inserted into the one and/or the other bifurcation branch of the vessel.

In accordance with the invention, the main catheter and/or at least one auxiliary catheter can comprise a lumen for guiding an imaging catheter of the imaging device. If necessary, only the auxiliary catheters can comprise a lumen for guiding an imaging catheter for optical coherence tomography. If only one auxiliary catheter is provided, both catheters, i.e. the main catheter and the auxiliary catheter, advantageously comprise lumina for accommodating and guiding an imaging catheter for optical coherence tomography, which can be correspondingly advanced into the bifurcation region of interest for the recording and records images through the wall of the optically transparent catheter in this region for coherence tomography. Instead of the catheter for optical coherence tomography, other imaging catheters and/or other imaging devices can be guided in corresponding lumina of the catheter if necessary, in order for instance to achieve an improved imaging in the case of exsanguinous vessels.

In accordance with the invention, the main catheter and/or at least one auxiliary catheter can be transparent at least in sections for light beams emitted on the part of an imaging device for optical coherence tomography. A material is thus used for the catheters, in the regions in which a subsequent imaging is intended, which is transparent for the wavelengths used for the imaging by means of optical coherence tomography, in the close infrared range for instance. A trouble-free image recording through the walls of the catheters is thus ensured in this way.

The main catheter and/or at least one auxiliary catheter can be designed as over-the-wire catheters and/or using another guidance technology. With the over-the-wire technology, the positioning is carried out using guide wires, with two guide wires being needed to position two auxiliary catheters. Different guidance technologies can also be used on the one hand for the main catheter, and on the other hand for one or more auxiliary catheters.

By designing the main catheter as an over-the-wire catheter, the guide wire for guiding a further occlusion balloon can be guided through a lumen of an auxiliary catheter. The guide wire thus simultaneously functions as a guide catheter for an auxiliary balloon.

The main catheter can comprise a lumen for the supply of a gaseous medium for inflating at least one balloon. The gas and/or gas mixture, for instance carbon dioxide or a mixture containing carbon dioxide, which is compatible intravascularly, is inserted into the corresponding lumen under pressure in order to inflate the main balloon and/or the further balloons. In this way it is possible to supply pressure to two balloons on a main catheter via a lumen for instance. The gas supply is advantageously realized with a total of one pump, with the use of several pumps however also being possible.

The main catheter can comprise at least one lumen for introducing a rinsing agent, in particular of gas and/or a fluid. The rinsing agent, such as for instance a saline solution or carbon dioxide, allows any blood which is present to be flushed away, in order to improve the imaging which is adversely affected by the scattering of blood components. If the lumen for the rinsing agent produces an opening which is distal to the proximal balloon, rinsing agents, such as for instance intravascularly compatible carbon dioxide or if necessary a fluid and/or a mixture of a gas with a fluid, can enter into the vascular region. By introducing the rinsing agent, the blood or a mixture of blood and fluid flows off distally through the vessel. By filling the distal balloon, a reverse flow of blood from the vascular branches is avoided and the seal needed for setting up a low pressure occlusion is achieved.

The lumen for introducing the rinsing agent can correspond to a lumen for an auxiliary catheter. If necessary, two auxiliary catheters are guided in the lumen, said auxiliary catheters being subsequently advanced into the two vascular branches. The auxiliary catheters then practically diverge from the main catheter, corresponding to the divergence of the auxiliary branches of the vessel from the main branch. The lumen for introducing the rinsing agent again ends in an opening, from which intravascularly compatible rinsing agent is filled up in the vascular region, whilst blood or a mixture of blood and fluid flows out through the still open vessel until the balloons of the auxiliary catheter(s) are occluded.

In accordance with the invention, the main catheter can comprise an opening for introducing a rinsing agent into the vessel, said opening arranged distal to the balloon for the proximal occlusion of the vessel. As previously described, the lumen for introducing the rinsing agent advantageously ends in this opening, so that the gas and/or the fluid for rinsing escapes here into the bifurcation region. The opening allows auxiliary catheters to be inserted into the bifurcation branches.

Advantageously, the balloons can be sequentially occluded. A sequential occlusion first enables the proximal balloon to be inflated and thus cuts off the blood supply from the main branch into the bifurcation region. It can then be rinsed in order to allow the blood to flow out into at least one bifurcation branch which is still open. Then, either both distal balloons are inflated or in the case whereby a distal balloon was inflated simultaneously with the proximal balloon, the remaining distal balloon is filled with pressure in order to occlude its vascular branch.

A pressure divider is advantageously provided with a valve system for sequential occlusion. This avoids the operation of several pumps, in that the individual exits of the pressure divider can be provided in each instance with valves and can be switched on. Stroke valves and/or controllable valves can be provided as valves. This enables a simple sequential occlusion. Naturally several pumps can be used instead or in addition in order to fill each balloon individually with pressure.

When the catheter device is completely occluded, the vessel is closed by approximately 90%, with rinsing fluid being used for the part which is not closed in order to ensure the mapping quality. A complete closure results in problems during the removal of the expendable catheter and can thus not be carried out without risking the patient. Nevertheless, the volume of the rinsing fluid and/or the rinsing gas which must be applied to the patient is significantly reduced by means of the catheter device according to the invention. This hereby reduces the risk of interrupting the blood supply because blood remaining in the capillaries is not rinsed out. In accordance with the invention, the adverse affects existing with previous catheter devices, despite rinsing, are avoided during imaging. The use of optical coherence tomography is thus also possible with larger vessels. The mapping preserves the geometric relationships, thereby advantageously increasing the usefulness of the diagnosis. An imaging catheter is additionally aligned in the vessel by means of the balloon catheters, thereby resulting in an improved imaging particularly with large vessels.

In accordance with the invention, at least one, in particular all balloons, can be provided with radiopaque markers and/or can be filled with a contrast means solution or consist of a radiopaque material. This ensures optimum visibility of the balloons in an angiographic image, thereby enabling an examination to be monitored and the image recordings to be evaluated with the knowledge of the location of the balloon.

Furthermore, the invention relates to a method for imaging in the bifurcation region of a vessel, in particular within the scope of optical coherence tomography, using a catheter device as previously described, comprising the following steps:
Positioning a main catheter with an occlusion balloon proximal to the bifurcation,
Positioning a first and second guide wire distal to the bifurcation in each one of the two vascular branches,
Positioning two auxiliary catheters with occlusion balloons distal to the bifurcation in one of the two vascular branches in each case,
Inflating the proximal occlusion balloon and positioning an imaging catheter distal to the proximal occlusion balloon,
Filling up with rinsing agent and then inflating the distal occlusion balloon and
Producing image recordings using the imaging catheter.

In this case, which is based on the use of two auxiliary catheters, a main catheter is first introduced into the vessel, whereupon guide wires are positioned in the two vascular branches distal to the bifurcation. These guide wires enable two auxiliary catheters with occlusion balloons to be inserted into the two vascular branches, with the auxiliary catheters being able to be guided for this purpose in a common lumen of the main catheter. The main catheter comprises an opening at its distal end, from which the auxiliary catheters issue into the vascular region on the one hand, in order to be inserted into the two bifurcation branches, from which, on the other hand, an injected rinsing agent can additionally issue. This occurs after the proximal occlusion balloon has been inflated and the wire for the optical coherence tomography or another imaging catheter has been correspondingly positioned. It is not until afterwards that the distal balloons of the two auxiliary catheters are inflated in the bifurcation branches, whereupon the imaging can be started.

Advantageously, the optimum balloon diameter can be selected for each vascular branch by means of different sizes of balloon. Furthermore, it is possible to adjust the size of the balloon for occluding the vessel by way of the applied pressure.

Furthermore, provision is made in accordance with the invention for a method for imaging in the bifurcation region of a vessel, in particular within the scope of optical coherence tomography, which, by using a catheter device as described previously, comprises the following steps:
Positioning a main catheter with an occlusion balloon in each instance proximal to the bifurcation, and in a vascular branch distal to said bifurcation,
Positioning an auxiliary catheter with an occlusion balloon distal to the bifurcation in the other vascular branch,
Positioning an imaging catheter distal to the proximal occlusion balloon,
Inflating the proximal and distal occlusion balloon of the main catheter,
Filling up with rinsing agent and then inflating the distal occlusion balloon of the auxiliary catheter and
Producing image recordings using the imaging catheter.

In this way, the main catheter is designed as a double balloon catheter, which additionally comprises a lumen for an auxiliary catheter, which ends in an opening between the two balloons which are arranged proximal and distal to the branching, advantageously in front of the bifurcation. An imaging catheter is then positioned in a suitable manner. The auxiliary catheter can leave the main catheter through this opening and can close a bifurcation branch. When the proximal and distal balloons of the main catheter are inflated at the same time, the blood supply is interrupted and the reflux from a side branch is prevented. It is alternatively also possible to inflate the two balloons sequentially. A rinsing agent inserted through the opening then displaces the blood, which flows out through the vascular branch which is still open, whereupon the balloon of the auxiliary catheter can likewise be inflated and the associated vascular branch occluded. The sequence of the method steps can be temporally changed, so if necessary it is possible for the imaging catheter not to be positioned until after the balloon of the main catheter has been inflated for instance.

### BRIEF DESCRIPTION OF THE DRAWINGS

Further advantages, features and details of the invention result from the following exemplary embodiments as well as on the basis of the drawings, in which:

FIG. 1A shows a catheter device according to the invention with a main catheter and two auxiliary catheters,

FIG. 1B shows a cross-section through an auxiliary catheter in FIG. 1A,

FIG. 1C shows a cross-section through the main catheter in FIG. 1A,

FIG. 2A shows a catheter device according to the invention with a main catheter and an auxiliary catheter,

FIG. 2B shows a cross-section through the auxiliary catheter in FIG. 2A,

FIG. 2C shows a cross-section through the main catheter in FIG. 2A,

FIG. 3 shows a pressure divider system for a catheter device according to FIG. 1A,

FIG. 4 shows a pressure divider system for a catheter device according to FIG. 2A,

FIG. 5 shows a flow diagram for a method according to the invention using a catheter device according to FIG. 1A and

FIG. 6 shows a flow diagram for a method according to the invention using a catheter device according to FIG. 2A.

### DETAILED DESCRIPTION OF THE INVENTION

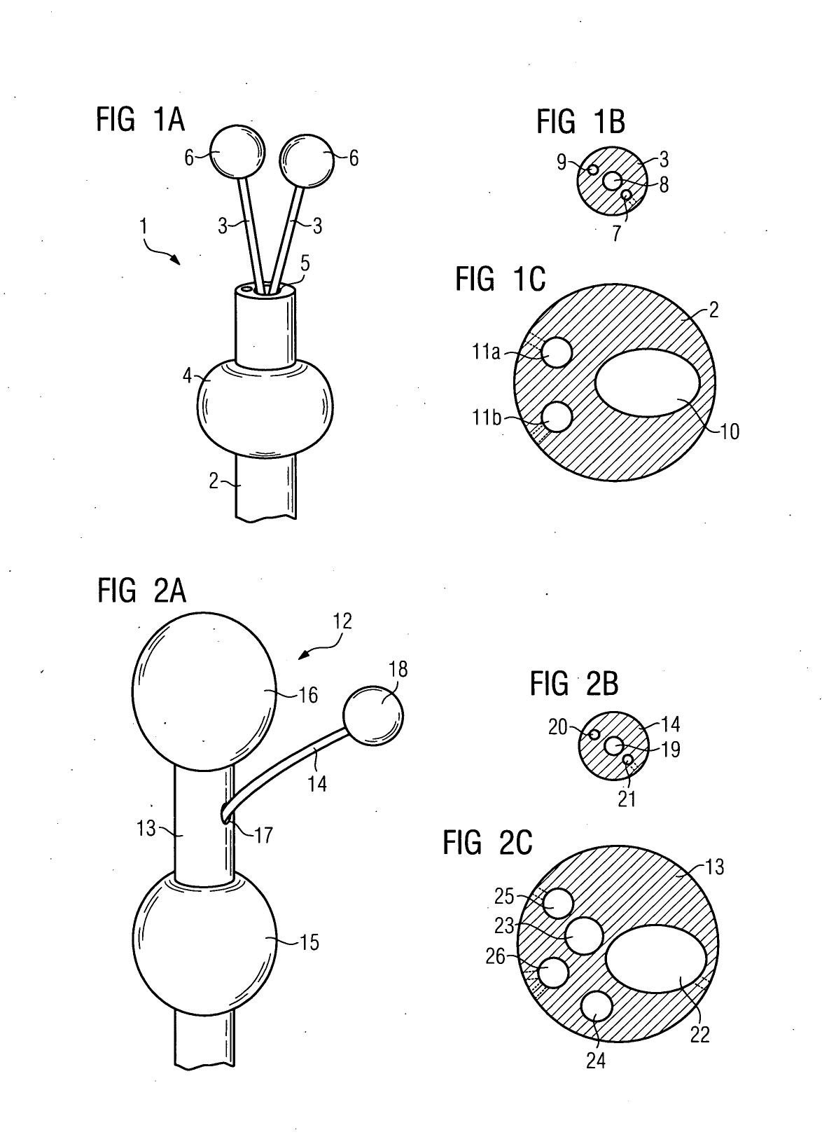

FIG. 1A shows a catheter device 1 according to the invention having a main catheter 2 and two auxiliary catheters 3. The main catheter 2 comprises a balloon 4, with which a vessel proximal to a bifurcation can be closed. The balloon 4 is shown inflated in the illustration. The two auxiliary catheters 3 issue through an opening 5 in the main catheter 2, said auxiliary catheters 3 having for their part distal occlusion balloons 6, with which the bifurcation branches can be closed. To this end, the auxiliary catheters 3 are inserted into the auxiliary branches of the bifurcation, at a specific angle to the main catheter 2, said angle being predetermined by the vascular geometry. In addition, the opening 5 comprises a region, through which a rinsing agent can be introduced into the vessel. A low pressure occlusion of the vessel comprising a bifurcation is possible by means of balloons 4 and/or 6. This enables an imaging for instance within the scope of optical coherence tomography without interference due to a retrograde blood flow from one or both of the vascular branches on the other side of the bifurcation.

FIG. 1B shows a cross-section through an auxiliary catheter 3 in FIG. 1A. The auxiliary catheter additionally comprises a lumen 7 for filling the distal balloon 6 assigned to the auxiliary catheter 3 via a further lumen 8, which is designed to accommodate an imaging catheter. A third lumen 9 is designed to accommodate a guide wire. The auxiliary catheter 3 is guided using monorail technology, in other words in an over-the-wire technology, in which only the distal catheter part is guided by the wire.

A cross-section through the main catheter 2 can be seen in FIG. 1C. The main catheter 2 comprises a large lumen 10 for accommodating the two auxiliary catheters 3. A second lumen 11a is provided separately from the above, said lumen serving to introduce a rinsing agent into the intermediate region between the proximal and the distal balloons 4 and/or 6. Furthermore, the cross-section of the main catheter 2 shown here illustrates a third lumen 11b, which is provided proximal to the proximal occlusion balloon and is used to supply a gaseous medium for inflating said proximal balloon.

By using two auxiliary catheters 3, each with their own distal occlusion balloon 6, it is possible to optimally select the balloon diameter for each of the vascular branches, in order to achieve the desired occlusion.

FIG. 2A shows a catheter device 12 according to the invention with a main catheter 13 and an auxiliary catheter 14, with the main catheter 13 having a proximal balloon 15 and a distal balloon 16, the occlusion balloons of which are both shown here in an inflated state. An opening 17 is provided between the proximal balloon 15 and the distal balloon 16 of the main catheter 13, through which opening an auxiliary catheter 14 with its own distal balloon 18 can issue. The distal balloon 18 of the auxiliary catheter 14 for occluding a vascular branch is likewise shown in an inflated state.

With the catheter device 12 shown here, the proximal balloon 15 and the distal balloon 16 of the main catheter 13 are approximately of the same size, whereas the distal balloon 18 of the auxiliary catheter 14 is inflated significantly less. Accordingly, the illustrated catheter device 12 lends itself to use with bifurcations in which the main branch crosses over into a bifurcation branch of approximately the same size, whereas the second bifurcation branch is less developed, so that an occlusion can be achieved by means of the distal balloon 18 of the auxiliary catheter 14. The backflow of blood and the retrograde reflux from the two vascular branches is prevented by blocking proximal to the bifurcation and distal in both bifurcation branches.

FIG. 2B shows a cross-section through the auxiliary catheter 14 in FIG. 2A. In a central region, this comprises a lumen 19 for accommodating an imaging catheter, so that this can be advanced in a transparent region between the balloon 15 and/or 16 and 18. Furthermore, a lumen 20 is provided for guiding the auxiliary catheter 14, which is designed as a monorail guide. A third lumen 21 of the auxiliary catheter 14 allows the supply of a gaseous medium for inflating the distal balloon 18 of the auxiliary catheter 14.

The main catheter 13 comprises the cross-section shown in FIG. 2C proximal to the first, proximal occlusion balloon 15. The auxiliary catheter 14 is guided via a larger lumen 22, whilst an essentially central lumen 23 for guiding an imaging catheter is again provided for optical coherence tomography for instance. The guiding is carried out via a lumen 24 by means of a guide wire using over-the-wire technology and/or as a monorail guide. In monorail technology, which represents one variant of over-the-wire technology, only the distal part of the balloon catheter is guided by the centrally arranged wire. With over-the-wire technology, the balloon is introduced using the guide wire as a guide rail.

Furthermore, the main catheter 13 has a lumen 25 for introducing a rinsing agent such as a gas and/or a fluid into the region between the proximal balloon 15 and the distal balloon 16 and 18. The filling of the proximal balloon 15 as well as the distal balloon 16 is enabled with the aid of the lumen 26, via which a gaseous medium to inflate the balloon 15, 16 is filled. The balloons 15, 16 can thus be filled at the same time, whereas rinsing of the vascular region lying therebetween is subsequently carried out via the lumen 25, whereupon the distal balloon 18 of the auxiliary catheter 14 is inflated for occluding the remaining vascular branch.

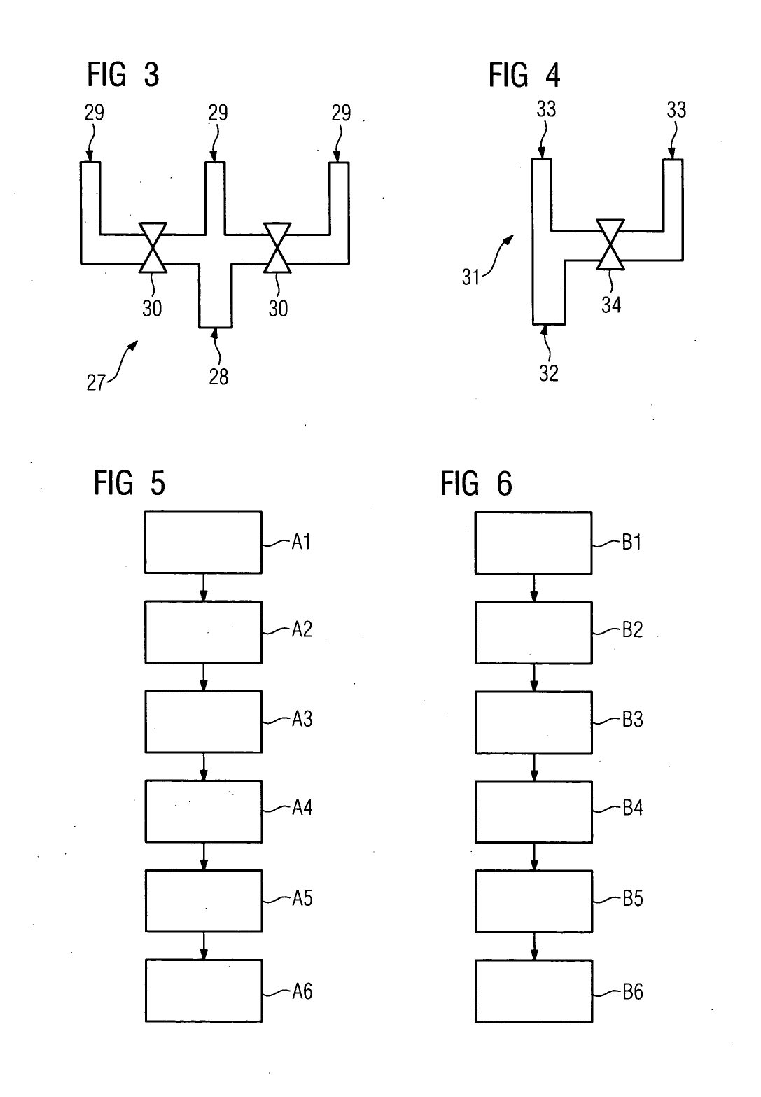

FIG. 3 shows a pressure divider system 27 for a catheter device according to FIG. 1A. In this system, a pressure inlet opening 28 faced by three pressure outlet openings 29 is provided in the center, said three pressure outlet openings 29 each being assigned to the two auxiliary catheters and to the main catheter. The pressure outlet openings 29 of the main catheter and of an auxiliary catheter can be closed in each instance via valves 30. A sequential occlusion of the individual balloons using just one pump is thus enabled.

A pressure divider system 31, which is shown in FIG. 4, is provided for a catheter device, according to FIG. 2A, which comprises a main catheter and an auxiliary catheter. Here two pressure outlet openings 33 for the main catheter and/or the auxiliary catheter face the pressure inlet opening 32. The second exit with the pressure outlet opening 33 for the auxiliary catheter can be switched on via a valve 34, so that it is first possible to inflate the proximal and distal balloon of the main catheter and only then, after rinsing, to fill the distal balloon in the remaining side branch with gas in order to occlude said side branch.

FIG. 5 shows a flow diagram for a method according to the invention using a catheter device 1 according to FIG. 1A. In this way, in a step A1, the main catheter with an occlusion balloon is first positioned proximal to the bifurcation, whereupon in step A2, a first and second guide wire are arranged distal to the bifurcation in one of the two vascular branches of the bifurcation in each case.

It is then possible in step A3, to position the two auxiliary catheters with their occlusion balloons distal to the bifurcation in one of the two vascular branches in each case. Then the proximal occlusion balloon of the main catheter is inflated in step A4, and the imaging catheter is positioned distal to the proximal occlusion balloon. Subsequently in a step A5, a rinsing agent such as carbon dioxide can be filled, and the distal occlusion balloon of the two auxiliary catheters can then be inflated. During the inflation, further rinsing is subsequently carried out, if necessary with a reduced volume. The production of image recordings using the imaging catheter is subsequently provided in step A6, for which purpose the main catheter and the auxiliary catheters in the regions in which the imaging is carried out, consist of a material which is transparent and suitable for this purpose.

A flow diagram for a method according to the invention using a catheter device 12 according to FIG. 2A is shown in FIG. 6. In this way, a main catheter with two occlusion balloons is first positioned in step B1, one of said two occlusion balloons being arranged proximal and the other distal to the bifurcation. In step B2, an auxiliary catheter with an occlusion balloon is then positioned distal to the bifurcation in the other vascular branch, for which purpose the main catheter comprises a corresponding opening out of which the main catheter can issue. In step B3, the imaging catheter for the imaging method is positioned distal to the proximal occlusion balloon of the main catheter. In step B4, the proximal distal occlusion balloon of the main catheter is inflated, with this being able to be carried out if necessary prior to positioning the imaging catheter. A rinsing agent is injected and/or filled in step B5 only after the occlusion balloons of the main catheter are inflated, in order to allow the blood and/or a mixture of blood and fluid to flow out of the vascular branch which is still open. In step B6, the desired image recordings are finally produced using the imaging catheter, said image recordings being able to be produced with a good image quality, despite a reduced quantity of the introduced rinsing agent, by preventing the retrograde blood flow from flowing out of the side branch.

1.-17. (canceled)

18. A catheter device which inserts into a bifurcation region of a vessel of a live body, comprising:
a first balloon arranged on a main catheter and positioned in a main vessel of the bifurcation for occluding a proximal area of the bifurcation; and
two further balloons each positioned in one of two vessel branches at a distal area of the bifurcation, the two further balloons each arranged on the main catheter and an auxiliary catheter respectively at a distance from the first balloon for occluding the distal area of the bifurcation.

19. The catheter device as claimed in claim 18, wherein the catheter device is used in an optical coherence tomography.

20. The catheter device as claimed in claim 18, wherein the two further balloons are arranged on the auxiliary catheter and a further auxiliary catheter respectively.

21. The catheter device as claimed in claim 20, wherein the two auxiliary catheters are guided in:
two different lumens respectively in a lumen of the main catheter, or
a common lumen in the lumen of the main catheter.

22. The catheter device as claimed in claim 18, wherein the two further balloons are arranged on bifurcation branches of the main catheter.

23. The catheter device as claimed in claim 18, wherein the main catheter or the auxiliary catheter comprises a lumen for guiding an imaging catheter of an imaging device.

24. The catheter device as claimed in claim 23, wherein the main catheter or the auxiliary catheter is transparent in a section for a light beam emitted from the imaging device for optical coherence tomography.

25. The catheter device as claimed in claim 18, wherein the main catheter or the auxiliary catheter is an over-the-wire catheter.

26. The catheter device as claimed in claim 25, wherein the guide wire for guiding the further occlusion balloons extends through a lumen of the auxiliary catheter.

27. The catheter device as claimed in claim 18, wherein the main catheter or the auxiliary catheter comprises a lumen for supplying a gaseous medium for inflating the first or the further balloons.

28. The catheter device as claimed in claim 18, wherein the main catheter comprises a lumen for introducing a rinsing agent.

29. The catheter device as claimed in claim 28, wherein the lumen for introducing the rinsing agent corresponds to a lumen for the auxiliary catheter.

30. The catheter device as claimed in claim 18, wherein the main catheter comprises an opening for introducing a rinsing agent.

31. The catheter device as claimed in claim 18, wherein the first and the two further balloons are sequentially inflated for a sequential occlusion of the vessel.

32. The catheter device as claimed in claim 31, wherein a pressure divider is provided with a valve system for the sequential occlusion.

33. The catheter device as claimed in claim 18, wherein the first balloon or one of the two further balloons is provided with a radiopaque marker or is filled with a contrast medium or consists of a radiopaque material.

34. A catheter device which inserts into a bifurcation region of a vessel of a live body, comprising:
a first balloon arranged on a main catheter and positioned in a main vessel of the bifurcation for occluding a proximal area of the bifurcation; and
two further balloons each positioned in one of two vessel branches at a distal area of the bifurcation, the two further balloons each arranged on an auxiliary catheter respectively at a distance from the first balloon for occluding the distal area of the bifurcation.

35. A method for generating an image recording in a bifurcation region of a vessel of a live body in a medical procedure using an insertable image catheter, comprising:
arranging a first balloon on a main catheter positioned in a main vessel branch at a proximal area to the bifurcation;
arranging two further balloons each on the main catheter and an auxiliary catheter respectively, the two further balloons each positioned in one of two vascular branches at a distal area to the bifurcation;
inserting the imaging catheter;
inflating the first and the two further balloons;
introducing a rinsing agent into the vessel; and
generating the image recording.

36. The method as claimed in claim 35, wherein the two further balloons are arranged on the auxiliary catheter and a further auxiliary catheter respectively.

37. The method as claimed in claim 35, wherein the first and the two further balloons are inflated sequentially.
